(19) United States
(12) Patent Application Publication
(10) Pub. No.: US 2021/0332419 A1
(43) Pub. Date: Oct. 28, 2021

(54) MONITORING HOST CELL CONTAMINATION OF VIRUS-BASED BIOLOGICAL PRODUCTS

(71) Applicant: Adaptive Phage Therapeutics, Inc., Gaithersburg, MD (US)

(72) Inventor: Carl Merril, Bethesda, MD (US)

(73) Assignee: Adaptive Phage Therapeutics, Inc., Gaithersburg, MD (US)

(21) Appl. No.: 17/279,797

(22) PCT Filed: Sep. 25, 2019
(86) PCT No.: PCT/US19/52987

§ 371 Date: Mar. 25, 2021

Related U.S. Application Data
(60) Provisional application No. 62/736,665, filed on Sep. 26, 2018.

(51) Int. Cl. C12Q 1/689; G01N 33/569

(52) U.S. Cl. CPC C12Q 1/689; G01N 33/56911

(57) ABSTRACT

The invention relates to the production of biopharmaceuticals and, more particularly, to a novel method of monitoring for contamination of such products with components of any host cells used or involved in the manufacturing process. The method comprises a step of determining the presence in a biopharmaceutical (e.g. a therapeutic phage composition) of a ribosomal subunit protein unique to bacteria (or protein fragments thereof) or a nucleic acid molecule comprising a nucleotide sequence derived from a gene encoding a ribosomal subunit protein unique to bacteria.

### BACKGROUND OF THE INVENTION

### Field of the Invention

The invention relates to the production of biopharmaceuticals and, more particularly, to a novel method of monitoring for contamination of such products with components of any host cells used or involved in the manufacturing process.

### Discussion of the Related Art

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Biopharmaceuticals, also known as biologic(al) medical products, biologicals, or more simply, biologics, are a diverse range of drug products produced from biological sources (e.g. they are manufactured in, extracted from, or semi-synthesized from biological sources). Already accounting for some 200+billion dollars in worldwide sales, it has been estimated that the total value of the global biophamaceuticals market will expand to a value of nearly 500 billion dollars by 2024 (Transparency Market Research report: Biologics Market-Global Industry Analysis, Size, Share, Growth, Trends, and Forecast 2016-2024).

One major class of biopharmaceuticals is directed to products which are based on viruses or viral components. These range from antibacterial viral agents (i.e. bacteriophages or “phages”), to viral vectors for the delivery of anticancer therapies, virally-encoded enzymes used to lyse specific pathogens (e.g. infective bacteria), and virus-based vaccines (e.g. inactivated or attenuated viral vaccines and viral subunit vaccines). These kinds of products are termed herein as “virus-based biological products”.

In order to ensure efficacy and safety of virus-based biological products, it is important that the manufacturing process is designed to, and/or steps are taken to, minimize contamination by components originating from any host cells used or involved in the manufacturing process. Such host cell components include, for example, cellular debris (which are potentially inflammatory) and more specific deleterious materials such as exo- and endo-toxins. Accordingly, it is typical for manufacturing processes of virus-based biological products to include a range of appropriate testing (e.g. the limulous amoebocyte lysate test (LAL test) for endotoxins, and suitable PCR and ELISA assays for contaminant host cell nucleic acid and proteins) and removal of host cell components where necessary (nb. residual levels up to 10 ng of host cell DNA per dose and 100 ppm of host cell protein are considered as generally acceptable in biopharmaceuticals).

However, many virus-based biological products require the use of specific cellular hosts for their production and as a consequence, testing and monitoring for all of the possible contaminating host cell components would be prohibitively time consuming and expensive. For example, for the production of the large numbers of specific “narrow host range” bacteriophage strains needed to treat a range of bacterial infections by “phage therapy” (Doss J et al., Viruses 9(3):50, 2017), wherein each strain has its own host cell requirements, would require a vast number of different testing assays to effectively monitor for host cell contaminants across the suite of required phage preparations. In addition, many, if not all, viruses share structural genes and their encoded proteins with their host cells, meaning that the use of any assay relying on the detection of general host cell proteins to monitor for “cellular contamination” is, in principle, flawed by the fact that the viruses themselves may contain proteins and genes that are identical to those of their host cells. Some examples of such gene- and protein-sharing between viruses and their host cells include:
Some phage tail proteins and bacterial pyocins (e.g. the R-type pyocin of Pseudomonas aeruginosa is related to a protein in the P2 phage, and the F-type pyocin is related to a protein of the lambda (λ) phage (Nakayama K et al., Mol Microbiol 38:213-231, 2000));
The bacterial T6SS apparatus (which is analogous to an upside-down bacteriophage tail complex) comprises a VgrG protein which is superimposable onto the “tail spike” gp27 and gp₅ proteins of the Escherichia coli bacteriophage T₄ (Pukatzki S et al., Proc Natl Acad Sci U.S.A. 104:15508-15513, 2007; and Mongous J D et al., Science 312:1526-1530, 2006);
The genes encoding the various components of the T6SS apparatus are also commonly found in a single gene cluster (comprising about 15 conserved ORFs) in a wide range of pathogenic bacteria, including E. coli, Vibrio cholerae, P. aeruginosa, Agrobacterium tumefaciens and Rhizobium leguminosarum (Das S et al., In Silico Biol 3:287-300, 2003); and
In the mycobacteriophage strain Giles, there is a short DNA segment at the right end of its genome that has a nucleotide sequence which is 100% identical with a sequence within the gene for the metE protein of its bacterial host, Mycobacterium smegmatis (Morris P et al., J Bacteriol 190:2172-2182, 2008).

Thus, there is a need to identify a novel method for the determination of contamination of virus-based biological products by host cell components which preferably relies on one or more fundamental difference(s) between viruses (including bacteriophage) and their hosts, such that, desirably, the need to assay for contaminating components from specific host cells (or a group of host cells) is wholly or largely avoided.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the examples and defined in the appended claims.

The invention relates to a novel method of monitoring for contamination of a biopharmaceutical with components of any bacterial cell(s) used or involved in the manufacture of the biopharmaceutical, comprising a step of determining the presence in the biopharmaceutical of a ribosomal subunit protein unique to bacteria (or protein fragments thereof) or a nucleic acid molecule comprising a nucleotide sequence derived from a gene encoding a ribosomal subunit protein unique to bacteria.

The biopharmaceutical will ordinarily be expected to be free or substantially free of any bacterial host cell contamination; having been subjected to one or more steps for the removal of bacterial cell components where necessary. Thus, where it is determined, using the method, that the biopharmaceutical, or more typically a sample of the biopharmaceutical, includes one or more ribosomal subunit protein unique to bacteria (or protein fragments thereof) and/or one or more nucleic acid molecule comprising a nucleotide sequence derived from a gene encoding a ribosomal subunit protein unique to bacteria, then the method indicates that the biopharmaceutical contains bacterial cell contamination. Subsequently, the biopharmaceutical may be subjected to one or more remedial steps, if necessary, to remove or reduce the bacterial cell contamination that is present.

The biopharmaceutical may, for example, comprise a biologically active protein produced from a bacterial host cell culture (e.g. a recombinant protein that is commonly produced in host cells such as E. coli, or more preferably, comprise a virus-based biological product such as a virus-based vaccine, virally-encoded enzyme or viral vector). However, in a particular application of the invention, the method is used with a virus-based antibacterial agent (i.e. a phage composition), particularly a therapeutic phage composition intended for use in phage therapy.

The method may comprise a step of determining the presence of a ribosomal subunit protein unique to bacteria (or protein fragments thereof) which, for example, may comprise conducting an immunologically-based assay (i.e. an “immunoassay”), such as an ELISA type assay, designed to quantitatively or qualitatively detect the ribosomal subunit protein (or protein fragments thereof). Alternatively, or additionally, the method may comprise a step of determining the presence of a nucleic acid molecule comprising a nucleotide sequence derived from a gene encoding a ribosomal subunit protein unique to bacteria which, for example, may comprise conducting a nucleic acid amplification assay (e.g. a polymerase chain reaction (PCR)-based assay) designed to quantitatively or qualitatively detect the nucleic acid molecule. As understood by one or ordinary skill in the art, the nucleic acid molecule that may be detected may comprise DNA or RNA (e.g. messenger RNA (mRNA)).

In some preferred embodiments, the step of determining the presence of a ribosomal subunit protein unique to bacteria (or protein fragments thereof) comprises conducting an immunoassay for a ribosomal subunit protein selected from the bacterial S16 and S18 ribosomal proteins (or protein fragments thereof).

In some other preferred embodiments, the step of determining the presence of a nucleic acid molecule comprising a nucleotide sequence derived from a gene encoding a ribosomal subunit protein unique to bacteria comprises conducting a nucleic acid amplification assay for a nucleic acid molecule comprising a nucleotide sequence derived from a gene encoding a ribosomal subunit protein selected from the bacterial S16 and S18 ribosomal proteins which are considered to have an essential role in bacterial cellular functions.

The invention further relates to a kit comprising at least a container containing, or a solid support having disposed thereon, a reagent suitable for use in an assay for detecting a ribosomal subunit protein unique to bacteria (or protein fragments thereof) or a nucleic acid molecule comprising a nucleotide sequence derived from a gene encoding a ribosomal subunit protein unique to bacteria, and wherein the kit optionally includes instructions for use of the kit in the method of the invention.

### DETAILED DESCRIPTION

The objects and features of the invention can be better understood with reference to the following Detailed Description.

### Definitions

The following definitions are provided for specific terms which are used in the following written description.

The term “virus”, as understood by one of ordinary skill in the art, refers to a non-cellular infective agent that reproduces only in a suitable host cell (e.g. an animal or plant host cell, or in the case of bacteriophage (“phage”), a bacterial host).

As used in the specification and claims, the singular form “a”, “an” and “the”, include plural references unless the context clearly dictates otherwise. Also, the term “virus” mentioned above, may refer to a single virus or more than one virus unless the context clearly dictates otherwise. Similarly, and as understood by one of ordinary skill in the art, the more specific terms of “bacteriophage” and “phage” can be used to refer to a single phage or more than one phage.

The present invention can “comprise” (open ended) or “consist essentially of” the components of the present invention. As used herein, “comprising” means the elements recited, or their equivalent in structure or function, plus any other element or elements which are not recited. The terms “having” and “including” are also to be construed as open ended unless the context suggests otherwise.

The term “about” or “approximately” means within an acceptable range for the particular value as determined by one of ordinary skill in the art, which will depend in part on how the value is measured or determined (e.g. the limitations of the measurement system). For example, “about” can mean a range of up to 20%, preferably up to 10%, more preferably up to 5%, and more preferably still up to 1% of a given value. Alternatively, particularly with respect to biological systems or processes, the term “about” can mean within an order of magnitude, preferably within 5 fold, and more preferably within 2 fold, of a value. Unless otherwise stated, the term “about” means within an acceptable error range for the particular value, such as ±1-20%, preferably ±1-10% and more preferably ±1-5%. In even further embodiments, “about” should be understood to mean +/−5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by one of ordinary skill in the art. This includes, at very least, the degree of expected experimental error, technique error and instrument error for a given technique used to measure a value.

Where used herein, the term “and/or” when used in a list of two or more items means that any one of the listed characteristics can be present, or any combination of two or more of the listed characteristics can be present. For example, if a composition is described as containing characteristics A, B, and/or C, the composition can contain A feature alone; B alone; C alone; A and B in combination; A and C in combination; B and C in combination; or A, B, and C in combination.

As used herein, the term “determining” encompasses a wide variety of actions. For example, “determining” may include calculating, computing, processing, deriving, investigating, looking up (e.g. looking up in a table, a database or another data structure), ascertaining and the like. Also, “determining” may include receiving (e.g. receiving information), accessing (e.g. accessing data in a memory) and the like. Also, “determining” may include resolving, selecting, choosing, establishing and the like.

As used herein, “phage therapy” refers to any therapy to treat a bacterial infection or bacterial-caused disease, which may involve the administration to a subject requiring treatment (e.g. a patient) of one or more therapeutic composition that can be used to infect, kill or inhibit the growth of a bacterium, which comprises one or more viable phage as an antibacterial agent (e.g. a phage composition comprising one phage strain or a phage “cocktail”) and which may further comprise, or otherwise be administered in combination with a further therapeutic composition comprising, one or more antibiotics, one or more bactericides, and/or one or more other therapeutic molecules such as small molecules or biologics that have bactericidal activity. Where more than one therapeutic composition is involved in the phage therapy, then the compositions may have a different host range (e.g. one may have a broad host range and one may have a narrow host range, and/or one or more of the compositions may act synergistically with one another). Further, as understood by one of ordinary skill in the art, the therapeutic phage composition(s) used in a phage therapy will also typically comprise a range of inactive ingredients selected from a variety of conventional pharmaceutically acceptable excipients, carriers, buffers, and/or diluents. The term “pharmaceutically acceptable” is used to refer to a non-toxic material that is compatible with a biological system such as a cell, cell culture, tissue, or organism. Examples of pharmaceutically acceptable excipients, carriers, buffers, and/or diluents are familiar to one of ordinary skill in the art and can be found, e.g. in Remington's Pharmaceutical Sciences (latest edition), Mack Publishing Company, Easton, Pa. For example, pharmaceutically acceptable excipients include, but are not limited to, wetting or emulsifying agents, pH buffering substances, binders, stabilizers, preservatives, bulking agents, adsorbents, disinfectants, detergents, sugar alcohols, gelling or viscosity enhancing additives, flavoring agents, and colors. Pharmaceutically acceptable carriers include macromolecules such as proteins, polysaccharides, polylactic acids, polyglycolic acids, polymeric amino acids, amino acid copolymers, trehalose, lipid aggregates (such as oil droplets or liposomes), and inactive virus particles. Pharmaceutically acceptable diluents include, but are not limited to, water and saline.

The term “unique to bacteria” refers to a feature or property of bacteria (prokaryotes) that distinguishes bacteria from other life forms, namely archea and eukaryotes. As understood by one of ordinary skill in the art, such a feature or property need not be uniform across all bacteria; in some cases, the feature or property may be held by a single bacterial species (or even by one particular strain), while in other cases, the feature or property may be shared by a group of bacteria or may otherwise be ubiquitous to all bacteria. Still, in other cases, the feature or property may be considered ubiquitous to bacteria notwithstanding some variation. For example, there are no orthologues of the bacterial ribosomal subunit proteins S16 and S18 found in archea or eukaryotes, and accordingly, these ribosomal proteins are considered as being unique to bacteria. The S18 protein, at least, is also known to be widely present (if not ubiquitous) across many species of bacteria notwithstanding that some variants may exist across species and/or strains.

The Applicant has recognized that an assay based upon determining the presence of one or more bacterial ribosomal subunit protein (or of a nucleic acid molecule comprising a nucleotide sequence derived from a gene encoding the bacterial ribosomal subunit protein) in a biopharmaceutical may provide a viable and simple method for monitoring (e.g. detecting) contamination of the biopharmaceutical by host cell components, wherein the ribosomal subunit protein is unique to bacteria inasmuch as no orthologous protein is found in archea or eukaryotes, nor for that matter, in viruses (including phage) which rely on host cell ribosomes and protein synthesis to reproduce and do not therefore include gene(s) encoding ribosomal proteins within their genomes.

Thus, the invention relates to a method of monitoring for contamination of a biopharmaceutical with components of any bacterial cell(s) used or involved in the manufacture of the biopharmaceutical, comprising a step of determining the presence in the biopharmaceutical of a ribosomal subunit protein unique to bacteria (or protein fragments thereof) or a nucleic acid molecule comprising a nucleotide sequence derived from a gene encoding a ribosomal subunit protein unique to bacteria.

The biopharmaceutical may, for example, comprise a biologically active protein produced from a bacterial host cell culture (e.g. a recombinant protein such as those selected from hormones and growth factors (e.g. insulin and human growth hormone (hGH)), and those used in anti-tumor therapies (e.g. cytokines such as granulocyte colony stimulating factor (G-CSF), interleukin proteins (e.g. IL-2) and interferons (e.g. IFN-α)) commonly produced in host cells such as E. coli (Sanchez-Garcia L et al., Microb Cell Fact 15:33, 2016), or more preferably, comprise a virus-based biological product such as a virus-based vaccine, virally-encoded enzyme or viral vector. However, in a particular application of the invention, the method is used with a virus-based antibacterial agent (i.e. a phage composition), particularly a therapeutic phage composition intended for use in phage therapy.

Ordinarily, the biopharmaceutical will be expected to be free or substantially free of any bacterial cell contamination; having been subjected to one or more steps for the removal of bacterial cell components where necessary. Thus, for example, where the method is used for monitoring for contamination of a phage composition with components from the host cell culture from which it as been produced, the determination (i.e. detection) of the presence in the phage composition, or more typically a sample of the phage composition, of one or more ribosomal subunit protein unique to bacteria (or protein fragments thereof) and/or one or more nucleic acid molecule comprising a nucleotide sequence derived from a gene encoding the ribosomal subunit protein unique to bacteria, will be indicative of contamination by bacterial host cell components, particularly where detection has been achieved quantitatively and determined such that the host cell components are present in amounts in excess of that generally considered as acceptable (e.g. levels corresponding to 10 ng of host cell DNA per dose and 100 ppm of host cell protein).

In some embodiments, the method comprises a step of determining the presence of a ribosomal subunit protein unique to bacteria (or protein fragments thereof). Preferably, the ribosomal subunit protein is selected from the bacterial S16 and S18 ribosomal proteins. The determining step may involve detecting the full length proteins and/or fragments thereof that are sufficiently sized so as to still be considered as unique to bacteria. Such fragments may be, for example, at least ₃o amino acids in length.

The presence of the S16 and/or s18 proteins and/or fragments thereof is preferably determined by using an antibody or fragment thereof which specifically binds to the S16 or S18 proteins (e.g. antibodies, preferably monoclonal antibodies, which have been raised against and specifically bind to the S16 or s18 proteins, antibody fragments such as Fv, Fab and F(ab)₂ fragments that are capable of specifically binding to the S16 or S18 proteins, and recombinant antibodies such as single chain antibodies (eg scFV antibodies) that are capable of specifically binding to the S16 or S18 proteins). As used herein, the term “specific binding” and grammatical equivalents means that the antibody or fragment thereof should not bind substantially to (that is, substantially “cross-react” with) another peptide, polypeptide or substance that may be present in the biopharmaceutical or a sample thereof. As such, it would be expected that specifically bound S16 or S18 protein (or fragments thereof) will be bound by the antibody or fragment thereof with at least ₃ times higher, more preferably at least 10 times higher, and most preferably at least ₅o times higher affinity than any other relevant peptide, polypeptide or substance. The antibody or fragment thereof may be used in a suitable immunoassay for the S16 or s18 proteins.

Suitable immunoassays for determining the presence of the S16 or s18 protein may, for example, involve: (i) contacting a solid support comprising surface-bound primary “capture” antibodies for the particular ribosomal protein with a sample of the biopharmaceutical (e.g. phage composition) and thereafter (ii) quantitatively detecting the amount of the S16 or s18 protein which has become bound to the support. The solid support may be composed of any of the typical materials well known to one of ordinary skill in the art including, inter alia, commercially available column materials, polystyrene beads, latex beads, magnetic beads, colloid metal particles, glass and/or silicon chips and surfaces, nitrocellulose strips, membranes, sheets, duracytes, wells and walls of suitable reaction trays such as 96-well plates and other plates, plastic tubes etc. It is also contemplated to use “suspension arrays” (Nolan J P and LA Sklar, Trends Biotechnol 20(1):9-12, 2002), wherein a carrier such as a microbead or microsphere is present in suspension, and the array consists of different microbeads or microspheres, possibly labeled, carrying different capture antibodies (or fragments thereof) (e.g. to separately bind to the S16 and s18 proteins or specific variants thereof). Methods of producing such arrays, for example based on solid-phase chemistry and photo-labile protective groups, are well known to one of ordinary skill in the art (see, for example, U.S. Pat. No 5,744,305).

The amino acid sequence of the 516 ribosomal protein of E. coli is:

(SEQ ID NO: 1)
MVTIRLARHG AKKRPFYQVV VADSRNARNG RFIERVGFFN
PIASEKEEGT RLDLDRIAHW VGQGATISDR VAALIKEVNK
AA,

and the amino acid sequence of the E. coli S18 ribosomal protein is:

(SEQ ID NO: 2)
MARYFRRRKF CRFTAEGVQE IDYKDIATLK NYITESGKIV
PSRITGTRAK YQRQLARAIK RARYLSLLPY TDRHQ.

As the amino acid sequences SEQ ID NO: 1 and SEQ ID NO: 2 are highly conserved across bacterial species, an antibody (or antibody fragment) which specifically binds to either of these sequences will detect S16 and s18 ribosomal protein contaminants in biopharmaceuticals produced by a manufacturing process using or involving a wide variety of bacterial cells including, in particular, species that are relevant for the manufacture of therapeutic phage compositions such as E. coli, P. aeruginosa and Klebsiella pneumoniae.

In some embodiments, the method comprises a step of determining the presence of a nucleic acid molecule comprising a nucleotide sequence derived from a gene encoding a ribosomal subunit protein unique to bacteria, preferably, a ribosomal subunit protein selected from the bacterial S16 and S18 ribosomal proteins. The determining step may involve detecting a nucleic acid molecule (e.g. DNA or mRNA) comprising the full length nucleotide sequence (i.e. corresponding to the nucleotide sequence of the complete gene for the ribosomal subunit protein) or of a portion only (e.g. a portion of the gene comprising only non-coding sequence such as regulatory sequences, or a portion comprising coding sequence only, or otherwise, a portion comprising some non-coding sequence and some coding sequence). In any case, the nucleic acid molecule to be detected ought to be sufficiently sized so that its nucleotide sequence is clearly corresponding to a nucleotide sequence derived from a gene encoding a ribosomal subunit protein unique to bacteria. Such nucleic acid molecules may be, for example, at least 90 nucleotides in length.

The presence of a nucleic acid molecule comprising a nucleotide sequence derived from a gene encoding the S16 or s18 proteins is preferably determined by using a nucleic acid amplification assay (e.g. a polymerase chain reaction (PCR)-based assay) designed to quantitatively or qualitatively detect the nucleic acid molecule using, for example, oligonucleotide primers designed to specifically amplify a target nucleotide sequence clearly corresponding to a nucleotide sequence derived from a gene encoding the S16 or s18 proteins. The amplification may be performed using any of the methods well known to one of ordinary skill in the art. In some embodiments, the amplification is performed using a standard polymerase chain reaction (PCR) amplification method using a pair of oligonucleotide primers (i.e. first and second primer sequences) defining the 5′ and 3′ ends of a target nucleotide sequence. However, in some circumstances, it may be preferred to perform the amplification step using, for example, a “nested” PCR amplification method using a further, “outside”, pair of primers (i.e. first and second outside primer sequences). The design of primers suitable for use in an amplification that may be used in the method, may be in accordance with techniques and guidelines well known to one of ordinary skill in the art (e.g. as described in Sambrook J and D W Russell, Molecular Cloning: a laboratory manual. Cold Spring Harbor Press, Third Edition (2001) at Chapter 8 (particularly Table 8-3)). In any case, for the amplification, the nucleotide sequences of the primers are preferably selected such that amplicons generated during the amplification are in the range of 5o to 3000 nucleotides in length, more preferably 90 to 1500 nucleotides in length. Generally, the shorter the amplicon, the more rapidly the amplification can be completed. One or more of the primers may be labelled with an appropriate label (e.g. biotin, fluorescein derivatives (e.g. FITC), rhodamine derivatives (e.g. TAMRA), Cascade Blue, Lucifer yellow, 5-bromo-2-deoxyuridine (BrdU), dinitrophenol (DNP), digoxygenin (DIG), and short peptide label sequences (i.e. short peptides against which specific antibodies can be raised), to enable detection (e.g. visualisation) of the amplicons to achieve the “result” of the method. Optionally, the amplicons may be sequenced using any of the standard nucleic acid sequencing methods.

Notwithstanding the fact that the amino acid sequences SEQ ID NO: 1 and SEQ ID NO: 2 are highly conserved, it is anticipated that the genes for the S16 and S18 ribosomal proteins will show minor variations across bacterial species. By using nucleic acid amplification designed to specifically amplify a target nucleotide sequence within the gene that includes one or more nucleotide sequence variations (e.g. mutation or polymorphisms) characteristic of a particular bacterial species or strain (or particular group of bacteria), the method may be conducted in a “tailored” manner so as to specifically determine contamination of a biopharmaceutical with nucleic acid from a particular bacterial species or strain (or particular group of bacteria). This may be useful in the context of, for example, a therapeutic phage composition comprising “narrow host range” bacteriophage strains (wherein each strain has its own host cell requirements) to determine the particular source of any host cell contamination that is present (i.e. the method may enable the determination of which of the host cells used for the production of the various phage strains has contaminated the phage composition).

However, by using nucleic acid amplification designed to specifically amplify a target nucleotide sequence within a gene encoding the S16 or S18 proteins that is widely found across bacterial species (i.e. a highly conserved genomic sequence) enables the method to be conducted in a manner so as to determine contamination of a biopharmaceutical with nucleic acid from a wide variety of bacterial cells including, in particular, species that are relevant for the manufacture of therapeutic phage compositions such as E. coli, P. aeruginosa and K. pneumoniae. One example of a suitable target nucleotide sequence for use in such embodiments of the method is from the gene for the S18 protein and has the following nucleotide sequence:

(SEQ ID NO: 3)
TTCAAGAG ATCGACTATA AAGATATCGC TACGCTGAAA
AACTACATCA CCGAAAGCGG TAAGATTGTC CCAAGCCGTA
TCACCGGTAC CCGTGCAAAA TACCAGCGTC AGCTGGCTCG
CGCTATCAAA CGCGCTCGCT ACCTGTCCCT GCTG

In some embodiments, the method comprises both a step of determining the presence of a ribosomal subunit protein unique to bacteria (or protein fragments thereof) such as the bacterial S16 and S18 ribosomal proteins, and a step of determining the presence of a nucleic acid molecule comprising a nucleotide sequence derived from a gene encoding a ribosomal subunit protein unique to bacteria (such as the bacterial S16 and S18 ribosomal proteins).

After conducting the method of the invention, it is indicated that the biopharmaceutical is contaminated with host cell components, then the biopharmaceutical may be subjected to one or more remedial steps, if necessary, to remove or reduce the bacterial cell contamination present.

The invention further relates to a kit comprising at least a container containing, or a solid support having disposed thereon, a reagent for use in an assay for detecting a ribosomal subunit protein unique to bacteria (or protein fragments thereof) or a nucleic acid molecule comprising a nucleotide sequence derived from a gene encoding a ribosomal subunit protein unique to bacteria, and wherein the kit optionally includes instructions for use of the kit in the method of the invention. The reagent may be, for example, a suitable antibody or fragment thereof which specifically binds to the ribosomal subunit protein, or may comprise an oligonucleotide primer or pair of oligonucleotide primers designed to specifically amplify a target nucleotide sequence clearly corresponding to a nucleotide sequence derived from a gene encoding a ribosomal subunit protein unique to bacteria. The kit may further comprise control agents (e.g. purified bacterial S16 or s18 ribosomal proteins) as would be apparent to one of ordinary skill in the art.

Still further, the invention relates to a method of monitoring for contamination of a biopharmaceutical or bioproduct with components of any bacterial or eukaryotic cell(s) used or involved in the manufacture of the biopharmaceutical or bioproduct, comprising a step of determining the presence in the biopharmaceutical or bioproduct of a ribosomal subunit protein (or protein fragments thereof) comprising the following amino acid sequence:

(SEQ ID NO: 4)
PRQYKIPDWFLNRQKDV

and/or a nucleic acid molecule comprising a nucleotide sequence encoding the amino acid sequence of SEQ ID NO: 4.

This method may be particularly suited to monitoring for contamination with components of eukaryotic cell(s) such as yeast cells and mammalian cell lines (including human cell lines). As will be appreciated by one of ordinary skill in the art, such a method may employ reagents and techniques the same as or similar to those described above (e.g. antibodies (or fragments thereof) which specifically bind to the 17 amino acid sequence in an immunologically-based assay such as an ELISA, and/or primer molecules designed to specifically amplify a nucleic acid molecule comprising a nucleotide sequence encoding the 17 amino acid sequence using a nucleic acid amplification). Conveniently, the method may be conducted using a kit. A suitable kit may comprise at least a container containing, or a solid support having disposed thereon, a reagent for use in an assay for detecting a ribosomal subunit protein (or protein fragments thereof) comprising the amino acid sequence of SEQ ID NO: 4 and/or a nucleic acid molecule comprising a nucleotide sequence encoding the amino acid sequence of SEQ ID NO: 4, and optionally includes instructions for use of the kit in the method.

Although the invention herein has been described with reference to embodiments, it is to be understood that these embodiments, and examples provided herein, are merely illustrative of the principles and applications of the present invention. It is therefore to be understood that numerous modifications can be made to the illustrative embodiments and examples, and that other arrangements can be devised without departing from the spirit and scope of the present invention as defined by the appended claims. All patent applications, patents, literature and references cited herein are hereby incorporated by reference in their entirety.

### EXAMPLES

The invention will now be further illustrated with reference to the following examples. It will be appreciated that what follows is by way of example only and that modifications to detail may be made while still falling within the scope of the invention.

### Example 1

### An immunoassay to determine the presence of contaminating bacterial components in a virus-based biological product.

Exploiting a fundamental difference between viruses and bacteria, an assay was developed to determine the presence of bacterial host cell contamination in a model phage composition. That difference was based on the inability of all viruses to conduct essential life processes outside of their host cells; a deficit mainly due to their lack of a mechanism to produce proteins. That is, viruses lack ribosomes necessary to translate the 4-letter DNA or RNA code into proteins essential for life processes, and therefore rely on host cell ribosomes and protein synthesis to reproduce.

Ribosomes are comprised of two major components: a small ribosomal subunit (which functions to “read” mRNA transcripts) and a large ribosomal subunit (which links encoded amino acids via peptide bonds to form a polypeptide chain (i.e. a protein)). Each subunit comprises one or more ribosomal RNA (rRNA) molecules and a variety of ribosomal proteins (r-protein or rProtein). It has been recognized that ribosomal proteins are among the most highly conserved proteins across all life forms (Ban N et al., Curr Opin Struct Biol 24:165-169, 2014).

Among the 40 proteins found in the various small ribosomal subunits, 15 subunits are universally conserved across prokaryotes and eukaryotes. However, 7 subunits are only found in bacteria (namely, S21, S6, S16, S18, S20, S21 and THX), while 17 subunits are only found in archea and eukaryotes (Ban N et al., supra). Turning to the proteins found in the various large ribosomal subunits, in this case, 18 proteins are considered to be universal (i.e. found in bacteria, eukaryotes and archea), 14 proteins are only to be found in bacteria, and 27 proteins are only known to be found in archea and eukaryotes. The small and large ribosomal subunit proteins unique to bacteria thus represented a possible “target” analyte for the development of a viable and simple assay for monitoring the presence of bacterial host cell contamination in a viral-based biological product.

However, a further selection criterion was identified; namely that the bacterial ribosomal proteins should be restricted to any that have an essential role in bacterial cellular functions (e.g. ribosomal proteins that are essential for bacterial survival, viability and/or reproduction). The S18 ribosomal protein was consequently selected for further assessment and development of an assay. This protein is a highly conserved structural protein found in all eubacteria and, along with the S6 ribosomal protein, provides structural stability to the 30S ribosomal subunit. The S18 protein is also ubiquitous in bacterial ribosomal structures across many species of bacteria.

1,000 sequenced and annotated bacterial genomes stored in the National Center for Biotechnology Information (NCBI) databases were examined using the “Basic Local Alignment Search Tool (BLAST)” to confirm the highly conserved nature of the S18 protein. It was found that all of the examined isolates have identical S18 proteins with a 100% overlap in their amino acid sequences. Importantly, species relevant to the manufacture of therapeutic phage composition for a number of pathogenic bacteria, such as E. coli, P. aeruginosa, and K. pneumoniae all appeared in this collection. An additional 250 S18 bacterial proteins with 100% overlap in their amino acid sequence alignments, that have not yet been published, were also found using the Universal Protein Knowledgebase (UniProt) consortium database and search protocols. Further details are shown in Table 1 below.

TABLE 1

| | | | Range of | |
|---|---|---|---|---|
| | Status of | | Overlap in | |
| | Annotations | No. of | Amino acid | |
| Collection | in Collections | Matches | Sequences | WebLink |
| NCBI | Confirmed and | ~1000 | All identical | https://www.ncbi.nlm. |
| | annotated | | | nih.gov/ipg/NP_41862 |
| | | | | 3.1?log$=smartblast |
| UniProt | Annotations | 248 | All identical | https://www.uniprot.org/ |
| | under review | | | blast/uniprot/B2018 |
| | | | | 07118A530B6CA01 |
| | | | | 38AFAA6D2B97CE8 |
| | | | | C2A92421338F7 |

The amino acid sequence of the S18 protein (₇₅ amino acids in length) of E. coli found to be highly conserved in the abovementioned collections is as follows:

(SEQ ID NO: 2)
MARYFRRRKF CRFTAEGVQE IDYKDIATLK NYITESGKIV
PSRITGTRAK YQRQLARAIK RARYLSLLPY TDRHQ

The highly conserved nature of the S18 protein sequence across bacterial species indicates that the S18 protein is of fundamental importance to bacterial cellular functions. This provides considerable “comfort” in the selection of the bacterial S18 protein for the development of an assay to monitor for contamination of virus-based biological products by host cell proteins (e.g. by using an immunologically-based assay such as an ELISA).

The generation of antibodies suitable for the specific detection of bacterial S18 proteins have been described (Lelong J C et al., Proc Natl Acad Sci USA 71(2):248-252, 1974). Such antibodies could be employed in a variety of standard immunoassay formats, including well known ELISA type assays. For example, in one simple format, a “sandwich” ELISA format, anti-bacterial S18 ribosomal protein antibodies may be adsorbed onto a standard titre plate (eg a 96-well plate) as primary antibodies for the capture of any S18 ribosomal protein present in a suitable sample (e.g. an aliquot from a virus-based biological product such as a phage composition) applied to the plate. Following incubation to permit capture of any S18 protein, and any subsequent appropriate washing steps, any captured S18 ribosomal protein may be determined by use of secondary antibodies conjugated to an enzyme such as horseradish peroxidase (HRP) which enables detection by the generation of a chromagen from a supplied substrate.

### Example 2

A PCR assay to determine the presence of contaminating bacterial components in a virus-based biological product.

Given the highly conserved nature of the S18 protein sequence, an analysis was conducted to assess the level of variation in the nucleotide sequence of the encoding gene across bacterial species.

It was found that the most highly conserved bacterial genomic DNA sequence from the S18 protein gene comprises 152 nucleotides with the sequence:

(SEQ ID NO: 3)
TTCAAGAG ATCGACTATA AAGATATCGC TACGCTGAAA
AACTACATCA CCGAAAGCGG TAAGATTGTC CCAAGCCGTA
TCACCGGTAC CCGTGCAAAA TACCAGCGTC AGCTGGCTCG
CGCTATCAAA CGCGCTCGCT ACCTGTCCCT GCTG

This specific, highly conserved sequence encodes an amino acid sequence found in the S18 protein.

The recognition of this highly conserved genomic sequence thereby provides an alternative (and/or additional) means to monitor for host cell contamination of virus-based biological products. In particular, by assaying for host cell nucleic acid by using, for example, a PCR-based assay.

The design, synthesis and use of appropriate primer molecules in a PCR-based assay is well known and understood by one of ordinary skill in the art.

### Example 3

Prophetic example to determine the presence of contaminating bacterial ribosomal protein in a model composition for phage therapy comprising EcoIIIϕG phage.

A high titre composition comprising EcoIIϕG phage can be prepared following inoculation and culture of E. coli bacteria (EcoIII) in accordance with routine techniques well known to one of ordinary skill in the art.

For example, EcoIIϕG phage can be purified by Caesium chloride gradient in accordance with routine techniques. Here, the generated purified phage suspension (1 ml) can be precipitated with 10% polyethylene glycol 8000 (Sigma-Aldrich, St Louis, Mo., United States of America) and 0.5 M sodium chloride at 40° C. overnight. Subsequently, the suspension can be centrifuged at 17,700 g for 15 minutes and the supernatant removed. Alternatively, the phage suspension can be dialyzed. The PEG/salt-induced precipitate is resuspended in 0.5 ml of TE buffer (pH 9.0) and treated with 20 μl of 20 mg/ml proteinase K for 20 minutes at 56° C. followed by treatment with SDS at a final concentration of 2% at 65° C. for 20 minutes. This mixture is then phenol/chloroform (25:24:1 phenol:chloroform:isoamyl alcohol, Sigma Aldrich) treated at least twice and the aqueous phase is then precipitated with 2.5 volumes of ice cold 96% ethanol and 0.1 volume of sodium acetate (pH 4.8). Subsequent to centrifugation, the pellet is washed in 70% ethanol and resuspended in 100 μl of TE buffer (pH 8.0). Phage stocks can then be stored at 4° C. indefinitely. Phage titer can be assessed by plating ten-fold serial dilutions and calculating the plaque forming units (PFU).

The resultant phage composition can be considered to be a purified composition and it will be expected that the composition will therefore be substantially free of unwanted substances, including, but not limited to, proteins, nucleic acids, carbohydrates, lipids, subcellular organelles and/or other impurities (e.g. metals or other trace elements). The phage composition will also be of a high titre as may be suitable for phage therapy. As such, the phage composition may contain, for example, 10⁶ to 10¹⁴ plaque-forming units (PFU) of the phage.

Samples for testing for host cell contamination may be prepared as follows: a first phage sample can be prepared by taking a 1 mL aliquot of the phage composition by diluting 1:100 in stabilization media (SM) buffer. Further samples can be prepared in a similar manner and “spiked” with various amounts of recombinant E. coli S18 ribosomal protein (e.g. 0.01 μg, 0.1 μg, 1μg and 5 μg).

The samples are applied to separate wells of a 96-well plate (100 μl of the respective sample per well) to which anti-bacterial S18 ribosomal protein antibodies (primary “capture” antibodies) having been pre-adsorbed in accordance with standard techniques (e.g. by using a preparation of the antibodies at a concentration of 1-10 μg/ml in carbonate/bicarbonate buffer, pH 9.6, incubation overnight at 4° C., and subsequent washing (2×) of the plate with 200 μl per well of phosphate buffered solution (PBS)) with other remaining protein-binding sites blocked with an appropriate blocking solution (e.g. 5% non fat dry milk/PBS). The plate is then incubated at 37° C. for approximately 90 mins, the samples removed and the plate washed (2×; 200 μl per well of PBS). Thereafter, 100 μl of diluted secondary “detection” antibodies conjugated to HRP, which bind to a different S18 protein epitope to that recognised by the capture antibodies, is added to each well before incubation at room temperature (RT) for 1 to 2 hours. The plate is then washed (3-4×) with PBS. Detection is achieved by adding 100 μl per well of a substrate of HRP (e.g. 3,3′,5,5′-tetramethylbenzidine; TMB) to each well and incubating at RT for 15-30 mins. Optical density (OD) can then be read at 450 nm after adding a “stopping” solution (e.g. 2M H₂SO₄).

Only those samples including S18 ribosomal protein, either from the spiking or otherwise as the result of actual contamination of the phage composition, will provide a “positive” result. Accordingly, the present invention offers a novel method of determining contamination of biopharmaceuticals such as virus-based biological products (e.g. therapeutic phage compositions) by host cell components.

### Example 4

An immunoassay to determine the presence of contamination in a biopharmaceutical manufactured using host cells of animal origin.

Thought was given to the possibility of further developing the method of the invention such that it could not only be used for monitoring contamination of a biopharmaceutical with bacterial host cell components but additionally could be used for monitoring contamination of biopharmaceuticals produced using a manufacturing process using or involving any host cells of non-bacterial origin (e.g. eukaryotic host cells such as yeast and mammalian cell lines); that is, the method could also be applied for monitoring contamination of a biopharmaceutical with non-bacterial components.

To this end, a short amino acid sequence (17 amino acids in length) was identified within the protein sequence of the bacterial S13 ribosomal subunit that is conserved in over 100 different species of eukaryotes. The undertaken survey (using the Universal Protein Knowledgebase (UniProt) consortium Database search protocols) identified and highlighted the consistency of this 17 amino acid sequence in a divergent collection of eukaryotic organisms including ribosomal protein sequences from: mice, octopus, pythons, bats, manatees, fish and human (see the complete list of species examined in Table 2 below). The conserved 17 amino acid sequence is:

(SEQ ID NO: 4)
PRQYKIPDWFLNRQKDV

TABLE 2

| | | | |
|---|---|---|---|
| Python bivittatus | Clupea harengus | Anoplopoma fimbria | Gobiocypris rarus |
| Pseudopodoces humilis | Pongo abelil | Callorhinchus milii | Gillichthys seta |
| Nomascus leucogenys | Acipenser baerii | Propithecus coquereli | Branchiostoma floridae |
| Haliaeetus leucocephalus | Oncorhynchus mykiss | Macaca fascicularis | Camelus ferus |
| Escherichia coli | Sinocyclocheilus grahami | Labrus bergylta | Priapulus caudatus |
| Myotis davidii | Xenopus tropicalis | Homo sapiens | Stenella coeruleoalba |
| Aeromonas hydrophila | Elephantulus edwardii | Larimichthys crocea | Nematostella vectensis |
| Thamnophis sirtalis | Rhincodon typus | Oreochromis niloticus | Acanthaster planci |
| Trichechus manatus | Sinocyclocheilus | Odobenus rosmarus | Priapulus caudatus |
| latirostris | anshuiensis | divergens | Capitella teleta |
| Mus musculus | Protobothrops | Salmo salar | Argopecten irradians |
| Ailuropoda melanoleuca | mucrosquamatus | Marmota marmota marmota | Phragmatopoma lapidosa |
| Terrapene mexicana | Xenopus laevis | Hippocampus comes | Oryctolagus cuniculus |
| triunguis | Myotis brandtii | Mesocricetus auratus | SpadeIla cephaloptera |
| Sarcophilus harrisii | Paramormyrops kingsleyae | Leptonychotes weddellii | Lingula anatina |
| Apteryx australis mantelli | Danio rerio | Gambusia affinis | Octopus bimaculoides |
| Colobus angolensis palliatus | Takifugu rubripes | Eptesicus fuscus | Sipunculus nudus |
| Pteropus vampyrus | Esox lucius | Alligator sinensis | Crassostrea virginica |
| Pogona vitticeps | Nothobranchius furzeri | Macaca mulatta | Pomacea canaliculata |
| Sinocyclocheilus | Jaculus jaculus | Phoronis muelleri | Exaiptasia pallida |
| rhinocerous | Pteropus alecto | Pygocentrus nattereri | Bos mutus |
| Condylura cristata | Oryzias latipes | Ictalurus punctatus | |

As such, by targeting this conserved 17 amino acid sequence (or a nucleic acid molecule comprising a nucleotide sequence encoding the 17 amino acid sequence), a method for monitoring contamination of biopharmaceuticals with, for example, host cell components of any type (e.g. bacteria, yeast or mammalian cell lines (including human cell lines)) may be enabled. In addition, the method would be applicable to monitoring contamination of biopharmaceuticals and other bioproducts that may be isolated from materials of animal origin (e.g. bioactive peptides, lipoproteins and chitin from marine organisms). As will be appreciated by one of ordinary skill in the art, such a method may employ reagents and techniques the same as or similar to those described in the preceding examples (e.g. antibodies (or fragments thereof) which specifically bind to the 17 amino acid sequence in an immunologically-based assay such as an ELISA, and/or primer molecules designed to specifically amplify a nucleic acid molecule comprising a nucleotide sequence encoding the 17 amino acid sequence using a nucleic acid amplification).

The invention is not limited to the embodiment herein before described which may be varied in construction and detail without departing from the spirit of the invention. The entire teachings of any patents, patent applications or other publications referred to herein are incorporated by reference herein as if fully set forth herein.

What is claimed:

1. A method of monitoring for contamination of a biopharmaceutical with components of any bacterial cell(s) used or involved in the manufacture of the biopharmaceutical, comprising a step of determining the presence in the biopharmaceutical of a ribosomal subunit protein unique to bacteria (or protein fragments thereof) or a nucleic acid molecule comprising a nucleotide sequence derived from a gene encoding a ribosomal subunit protein unique to bacteria.

2. The method of claim 1, wherein the biopharmaceutical is a virus-based biological product.

3. The method of claim 2, wherein the virus-based biological product is a phage composition.

4. The method of claim 1, wherein the method comprises a step of determining the presence of a ribosomal subunit protein unique to bacteria (or protein fragments thereof), and wherein the ribosomal subunit protein is selected from the bacterial S16 and S18 ribosomal proteins.

5. The method of claim 4, wherein the presence of the S16 and/or s18 proteins (and/or fragments thereof) is determined by immunoassay.

6. The method of claim 1, wherein the method comprises a step of determining the presence of a ribosomal subunit protein unique to bacteria (or protein fragments thereof), and wherein the ribosomal subunit protein has the amino acid sequence of SEQ ID NO: 1 or SEQ ID NO: 2.

7. The method of claim 1, wherein the method comprises a step of determining the presence of a nucleic acid molecule comprising a nucleotide sequence derived from a gene encoding a ribosomal subunit protein unique to bacteria, and wherein the ribosomal subunit protein is selected from the bacterial S16 and S18 ribosomal proteins.

8. The method of claim 7, wherein the presence of a nucleic acid molecule comprising a nucleotide sequence derived from a gene encoding the S16 or S18 proteins is determined by a nucleic acid amplification assay.

9. The method of claim 8, wherein the nucleic acid molecule comprises the nucleotide sequence of SEQ ID NO: 3.

10. The method of claim 1, comprising both a step of determining the presence of a ribosomal subunit protein unique to bacteria (or protein fragments thereof), and a step of determining the presence of a nucleic acid molecule comprising a nucleotide sequence derived from a gene encoding a ribosomal subunit protein unique to bacteria.

11. A method of monitoring for contamination of a biopharmaceutical with components of any bacterial cell(s) used or involved in the manufacture of the biopharmaceutical, comprising a step of determining the presence of a ribosomal subunit protein selected from the bacterial S16 and S18 ribosomal proteins.

12. The method of claim 11, wherein the presence of the S16 and/or s18 proteins (and/or fragments thereof) is determined by immunoassay.

13. A method of monitoring for contamination of a biopharmaceutical with components of any bacterial cell(s) used or involved in the manufacture of the biopharmaceutical, comprising a step of determining the presence of a nucleic acid molecule comprising a nucleotide sequence derived from a gene encoding a ribosomal subunit protein selected from the bacterial S16 and S18 ribosomal proteins.

14. The method of claim 13, wherein the presence of a nucleic acid molecule comprising a nucleotide sequence derived from a gene encoding the S16 or S18 proteins is determined by a nucleic acid amplification assay.

15. A kit comprising at least a container containing, or a solid support having disposed thereon, a reagent for use in an assay for detecting a ribosomal subunit protein unique to bacteria (or protein fragments thereof) or a nucleic acid molecule comprising a nucleotide sequence derived from a gene encoding a ribosomal subunit protein unique to bacteria, and wherein the kit optionally includes instructions for use of the kit in the method of claim 1.

16. A method of monitoring for contamination of a biopharmaceutical or bioproduct with components of any bacterial or eukaryotic cell(s) used or involved in the manufacture of the biopharmaceutical or bioproduct, comprising a step of determining the presence in the biopharmaceutical or bioproduct of a ribosomal subunit protein (or protein fragments thereof) comprising the amino acid sequence of SEQ ID NO: 4 and/or a nucleic acid molecule comprising a nucleotide sequence encoding the amino acid sequence of SEQ ID NO: 4.

17. The method of claim 16, wherein the method is used to monitor for contamination with components of eukaryotic cell(s).

18. The method of claim 17, wherein the eukaryotic cell(s) are yeast or mammalian cells.

19. The method of claim 16, wherein the presence of the ribosomal subunit protein (and/or fragments thereof) is determined by immunoassay.

20. The method of claim 16, wherein the presence of a nucleic acid molecule comprising a nucleotide sequence encoding the amino acid sequence of SEQ ID NO: 4 is determined by a nucleic acid amplification assay.

21. A kit comprising at least a container containing, or a solid support having disposed thereon, a reagent for use in an assay for detecting a ribosomal subunit protein (or protein fragments thereof) comprising the amino acid sequence of SEQ ID NO: 4 and/or a nucleic acid molecule comprising a nucleotide sequence encoding the amino acid sequence of SEQ ID NO: 4, and wherein the kit optionally includes instructions for use of the kit in the method of any one of claimasil 16 to 20.


SEQUENCE LISTING (4 sequences)

SEQ ID NO: 1 - protein, 82 aa, Escherichia coli [sequence omitted]

SEQ ID NO: 2 - protein, 75 aa, Escherichia coli
MARYFRRRKFCRFTAEGVQEIDYKDIATLKNYITESGKIVPSRITGTRAKYQRQLARAIKRARYLSLLPYTDRHQ

SEQ ID NO: 3 - DNA, 152 bp, Escherichia coli
ttcaagagatcgactataaagatatcgctacgctgaaaaactacatcaccgaaagcggtaagattgtcccaagccgtatcaccggtacccgtgcaaaataccagcgtcagctggctcgcgctatcaaacgcgctcgctacctgtccctgctg

SEQ ID NO: 4 - protein, 17 aa, Artificial Sequence
  Synthetic Fragment
PRQYKIPDWFLNRQKDV